(19) United States
(12) Patent Application Publication
(10) Pub. No.: US 2025/0213793 A1
(43) Pub. Date: Jul. 3, 2025

(54) Automatic Injection Device

(71) Applicant: Becton Dickinson France, Le Pont-de-Claix (FR)

(72) Inventor: Franck Carrel, Saint Jean de Vaulx (FR)

(21) Appl. No.: 18/850,527

(22) PCT Filed: Mar. 24, 2023
(86) PCT No.: PCT/EP2023/057615

§ 371 Date: Sep. 24, 2024

(30) Foreign Application Priority Data
Mar. 25, 2022 (EP) 22305376.0

(51) Int. Cl. A61M 5/20; A61M 5/315; A61M 5/32

(52) U.S. Cl. CPC A61M 5/2033; A61M 5/3158; A61M 5/3204; A61M 2005/2026; A61M 2005/2073

(57) ABSTRACT

The disclosure relates to an automatic injection device including: a body; a medical container including a barrel containing a product for injection, a distal tip provided with an injection needle and a stopper in sliding engagement within the barrel. The medical container being slidably arranged in the body (10) between a proximal initial position and a distal position; a safety shield slidably mounted within the body between a distal initial position and a proximal second position; a plunger rod slidably arranged in the body. A non-circular proximal end releasably locked in a proximal initial position and a distal tip adapted to push the stopper in the distal direction when the proximal end is unlocked; a cam cooperating with the safety shield so that a translation of the safety shield from the distal initial position to the proximal second position causes the cam to rotate within the body to an unlocking position; a plunger retainer pivotally mounted in the body. A non-circular proximal opening arranged distally from the proximal end of the plunger rod in the initial position, said opening and the proximal end of the plunger rod having substantially complementary shapes, the plunger retainer being coupled to the cam so that, in the initial position, the shapes of the proximal end of the plunger rod and of the proximal opening are angularly offset from each other and, in the unlocking position, the shapes of the proximal end of the plunger rod and of the proximal opening are axially aligned, thereby allowing the plunger rod to move in the distal direction through the proximal opening of the plunger retainer.

### CROSS-REFERENCE TO RELATED APPLICATIONS

This application is the United States national phase of International Application No. PCT/EP2023/057615 filed Mar. 24, 2023, and claims priority to European Patent Application No. 22305376.0 filed Mar. 25, 2022, the disclosures of which are hereby incorporated by reference in their entireties.

### BACKGROUND OF THE INVENTION

### FIELD OF THE INVENTION

The present invention relates to a medical device for automatic injection of a product in a safe way, especially in emergency situations.

### DESCRIPTION OF RELATED ART

Some illnesses necessitate regular injections of drugs or products, for instance on a daily basis. In order to simplify the treatment, some self-injectors have been provided in order to allow the patient to perform the injection on his/her own.

Since the patient is usually neither a nurse nor an educated person in medical devices, such self-injectors must prove to be very simple to use and also very safe. In particular, the insertion of the needle must be performed at the right depth, the correct dose of product must be injected, that is to say a complete injection must be performed, and the injector must be deactivated after use before it is disposed of. Preferably, the needle should not be exposed, before and after use, in order to prevent any accidental needlestick injury.

Another important requirement of these self-injection devices is that they must not be able to be activated inadvertently, before the patient is ready to perform the injection, and in particular before the device is correctly applied at the right injection site.

Some automatic injection devices comprise a safety shield adapted to cover the needle tip except during injection, and a button to be pressed by the patient to trigger the injection.

The documents EP 2 921 191, WO 2021/067210 A1, US 2019/374728 A1 and WO 2009/040672 A2 disclose such an automatic injection device.

Before use, the safety shield and the needle are covered by a cap and the button is locked in order to avoid any accidental activation. The device comprises a mechanism configured to unlock the button once the safety shield has been pushed sufficiently onto the patient's skin.

Thus, the injection requires three actions from the user:
(1) removing the cap;
(2) pushing firmly the device onto the patient's skin to unlock the button; and
(3) pressing the unlocked button to activate the device and trigger injection.

However, when a drug has to be administered to the patient in an emergency situation, this three-step injection process may be too long or complex. For example, if a patient suffers from anaphylaxis, adrenaline has to be administered very quickly in order to avoid patient's death.

Thus, there is a need for self-injection devices that could be activated in a minimized number of steps without deteriorating the safety of the device. In particular, as indicated above, the device must not be able to be activated inadvertently and it also must avoid any accidental needlestick injury.

### SUMMARY OF THE INVENTION

The present invention meets this need by proposing a device for automatic injection of a product into an injection site, comprising:
a body;
a medical container comprising a barrel containing a product for injection, a distal tip provided with an injection needle and a stopper in sliding engagement within the barrel, the medical container being slidably arranged in the body between a proximal initial position and a distal position;
a safety shield slidably mounted within the body between a distal initial position and a proximal second position;
a plunger rod slidably arranged in the body, comprising a non-circular proximal end releasably locked in a proximal initial position and a distal tip adapted to push the stopper in the distal direction when the proximal end is unlocked;
a cam cooperating with the safety shield so that a translation of the safety shield from the distal initial position to the proximal second position causes the cam to rotate within the body to an unlocking position;
a plunger retainer pivotally mounted in the body, comprising a non-circular proximal opening arranged distally from the proximal end of the plunger rod in the initial position, said opening and the proximal end of the plunger rod having substantially complementary shapes, the plunger retainer being configured to cooperate with the cam so that, in the initial position, the shapes of the proximal end of the plunger rod and of the proximal opening are angularly offset from each other and, in the unlocking position, the shapes of the proximal end of the plunger rod and of the proximal opening are axially aligned, thereby allowing the plunger rod to move in the distal direction through the proximal opening of the plunger retainer.

Thanks to the cooperation between the cam and the plunger retainer, only two actions—which are: (1) removing the cap and (2) pressing the safety shield onto the patient's skin—are required from the user to fully perform the injection.

Thus, the ergonomics of the device is improved. Such improved ergonomics is in particular beneficial in emergency situations since it simplifies the operation of the device and may reduce the time required to inject the full dose to the patient.

In the present text, the distal end of a component or of a device is to be understood as meaning the end furthest from the user's hand and the proximal end is to be understood as meaning the end closest to the user's hand. Likewise, in the present text, the distal direction is to be understood as meaning the direction of injection, and the proximal direction is to be understood as meaning the opposite direction to the direction of injection.

In the present text, the term “axial” designates a direction parallel to the direction of injection (the direction of injection also corresponding to a longitudinal axis of the injection device) and the term “radial” designates a direction perpendicular to the direction of injection.

In some preferred embodiments, taken alone or in combination if appropriate:
the proximal end of the plunger rod comprises a plurality of teeth extending radially outwardly and the opening of the plunger retainer comprises a plurality of teeth extending radially inwardly such that the teeth of the plunger rod substantially fit respective recesses between the teeth of the opening of the plunger retainer such that, in the unlocking position, the proximal end of the plunger rod is allowed to pass through the opening of the plunger retainer;
the plunger rod comprises a non-circular anti-rotation portion arranged distally from the proximal end and the body comprises a non-circular central opening, said opening and the anti-rotation portion having substantially complementary shapes so that, in the initial position, the plunger rod is locked in rotation relative to the body by engagement of the anti-rotation portion in the central opening;
the cam comprises at least one recess adapted to receive a respective leg of the plunger retainer against an axial edge of the recess such that a rotation of the cam causes the plunger retainer to rotate;
the safety shield comprises at least one lug received in a respective first groove of the cam so that a movement of the safety shield in the proximal direction causes the cam to rotate;
the first groove presents an inclined first branch and an axial second branch connected to the first branch at a proximal end, such that, in the initial position, the lug is located at the distal end of the first branch and moves towards the proximal end of the first branch when the safety shield moves from the initial position to the second position;
the device comprises a first spring configured to urge the plunger rod in the distal direction;
the device comprises a second spring configured to urge the safety shield in the distal direction;
the device further comprises a ring supporting the medical container, the ring comprising at least one finger projecting radially outwardly, the cam further comprising at least one second groove receiving the respective at least one finger, the at least one second groove comprising a proximal portion, a distal portion and an inclined portion connecting the proximal portion to the distal portion, wherein the ring is locked at the proximal portion of the second groove and moves along the inclined portion to the distal portion when the plunger rod pushes the medical container from the initial position to the injection position;
an axial distance between the proximal portion and the distal portion of the second groove is equal to a penetration depth of the needle within the patient's body;
the at least one finger engages an axial groove of the body;
a translation of the ring in the distal direction causes the cam to rotate so that the lug of the safety shield engages the second branch of the first groove of the cam.

### BRIEF DESCRIPTION OF THE DRAWINGS

The terms Fig., FIG., Figs., FIGS., Figure, FIGURE, Figures, and FIGURES are used interchangeably to refer to the corresponding figures in the drawings.

Further features and advantages of the invention will be disclosed in the following detailed description, based on the appended drawings, wherein:

FIG. 1 is a perspective view of the injection device;

FIG. 2 is an exploded view of the body and the cap of the injection device;

FIG. 3 is an exploded view of the medical container and the needle shield of the injection device;

FIG. 4 is an exploded view of the plunger rod and the medical container;

FIG. 5 is a perspective view of the cap and the needle shield;

FIG. 6 is a perspective view of the medical container and the supporting ring;

FIG. 7 is a perspective view of the safety shield;

FIG. 8 is a perspective view of the cam;

FIG. 9 is a perspective view of the assembly of the safety shield, the cam and the ring;

FIG. 10 is an exploded view of the plunger rod and the plunger retainer;

FIG. 11 is a perspective view of the assembly of the plunger rod and the plunger retainer in the initial position;

FIG. 12 is a perspective view of the assembly of the safety shield, the cam and the plunger retainer;

FIG. 13 is a sectional view of the upper part of the injection device in the initial position;

FIGS. 14A and 14B are partially cut views of the plunger rod and the upper body in the initial position;

FIGS. 15A and 15B are top views of the plunger retainer and the plunger rod in the initial position and in the unlocking position, respectively.

### DESCRIPTION OF THE INVENTION

Unless otherwise specified, the components of the injection device are represented in their initial position, before use of the injection device. However, as will be explained below, some of the components are movable in translation and/or in rotation relative to each other to perform the injection. Thus, the description will refer not only to the initial position of the components, but also to one or more operative positions or phases that are reached during the injection process.

To summarize, before use, the body of the injection device is closed by a cap which protects the inside of the injection device from the outside environment.

In a first step, the cap is removed. As a result, the injection device is in an initial position. In said initial position, exposure of the needle and injection of the product are prevented by the fact that the needle and the medical container containing the product for injection are housed within the body of the injection device and that the plunger rod is locked in translation by a plunger retainer.

In a second step, to perform an injection, a safety shield is applied onto the patient's skin at an injection site. The safety shield is thus caused to translate in the proximal direction, which causes a cam coupled to the safety shield to rotate until a plunger retainer unlocks a plunger rod.

Said unlocking of the plunger rod triggers a continuous injection phase in which the plunger rod pushes a stopper in the distal direction. As a result, a medical container engaging the stopper is caused to translate in the distal direction-thereby allowing the needle to pierce the patient's skin at the right injection depth. Further, the movement of the stopper in the medical container in the distal direction expels the product from the medical container into the patient's body.

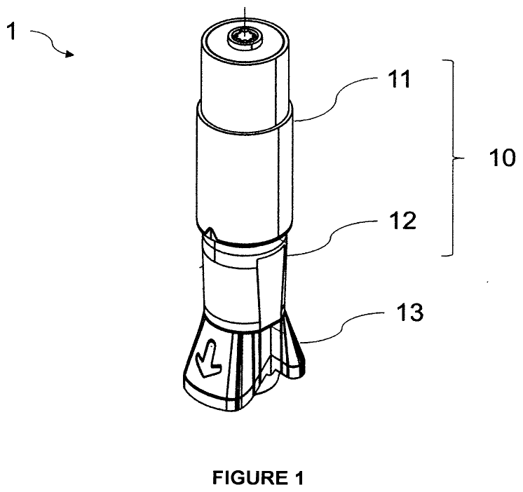

FIG. 1 shows a perspective view of a device for automatic injection according to an embodiment of the present disclosure and generally designated by reference number 1.

The device 1 comprises a housing 10 comprised of an upper body 11 and a lower body 12 that may be rigidly connected to each other.

The housing 10 has an outer shape adapted to be held in a user's hand. In general, the outer surface of the housing 10 is intended to be gripped by the palm and the fingers of the user's hand.

The connexion of the upper and lower bodies can be a snap-fit connection, screw-type connection, bayonet connection, or other means of connecting two parts together, in an unreleasable way or not. When the device is of a single use type, the means for connecting the upper body to the lower body are made unreachable to the user.

A cap 13 is removably connected to a distal end of the housing 10. The cap 13 can be connected to the lower body 12 by a snap-fit connection or by any other type of connection allowing removal of the cap 13 by an axial movement in the distal direction.

A medical container 20 such as, for example, a syringe, is received in at least one of the upper and lower bodies 11, 12.

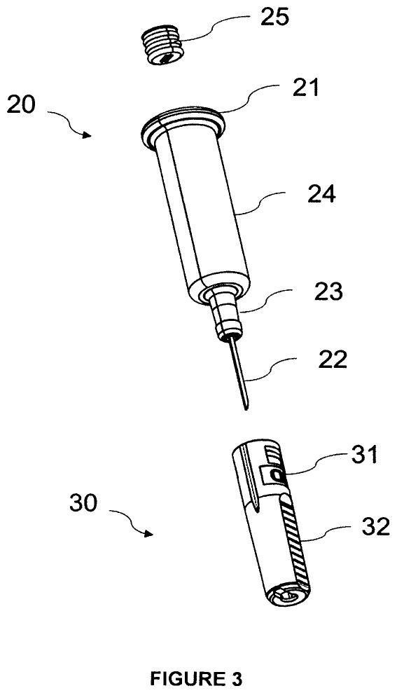

As shown in FIG. 3, the medical container 20 has a radial flange 21 defined at an open proximal end, and an injection needle 22 at a substantially closed distal end 23. Lateral walls extend between the proximal and distal ends and define a barrel 24 sized and shaped to contain a predetermined amount of a product for injection. The injection needle 22 may be fixed to the distal end 23, or removable therefrom, as a matter of design choice. The injection needle 22 is in fluid communication with the barrel 24 and provides an outlet port of the medical container 20 for the product.

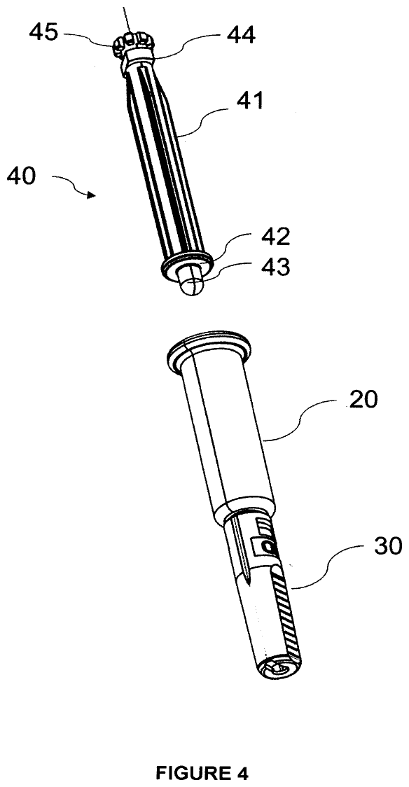

As shown in FIGS. 3 and 4, a needle shield 30 is provided at the distal end of the medical container 20 to cover and protect the needle 22 before use of the device 1. The needle shield 30 also provides for a sealing means of the distal end 23 of the medical container before use. To that end, the needle shield 30 may comprise an inner elastomeric shield 31 sealingly engaging the distal end of the medical container and an outer rigid cap 32 surrounding the inner shield.

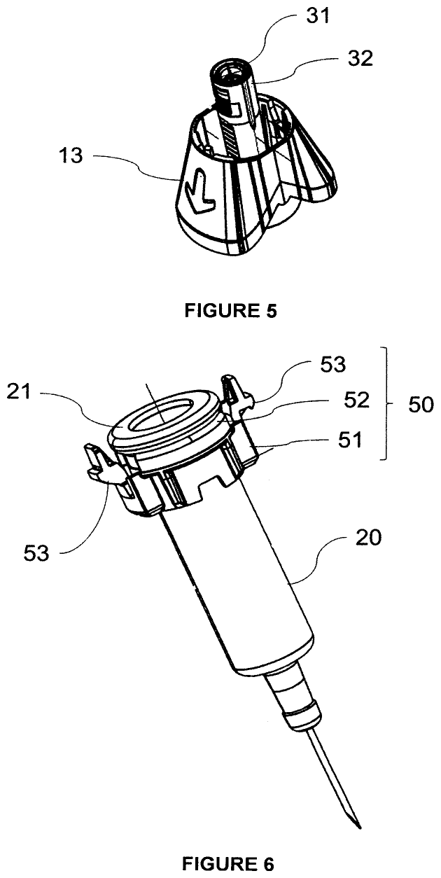

Before use (see FIG. 5), the cap 13 engages the outer rigid cap 32 so that removal of the cap 13 simultaneously removes the inner elastomeric shield 31 and the outer rigid cap 32 from the distal tip of the medical container.

Returning to FIG. 3, a stopper 25 is provided in the container 20 and is slidingly movable within the barrel 24. Movement of the stopper in the distal direction causes the product to be expelled from said medical container 20 through the injection needle 22 during the injection of the product into the patient. The stopper is typically made of an elastomeric material. The inner surface of the barrel and/or the outer surface of the stopper may be lubricated in order to reduce the gliding force of the stopper within the barrel.

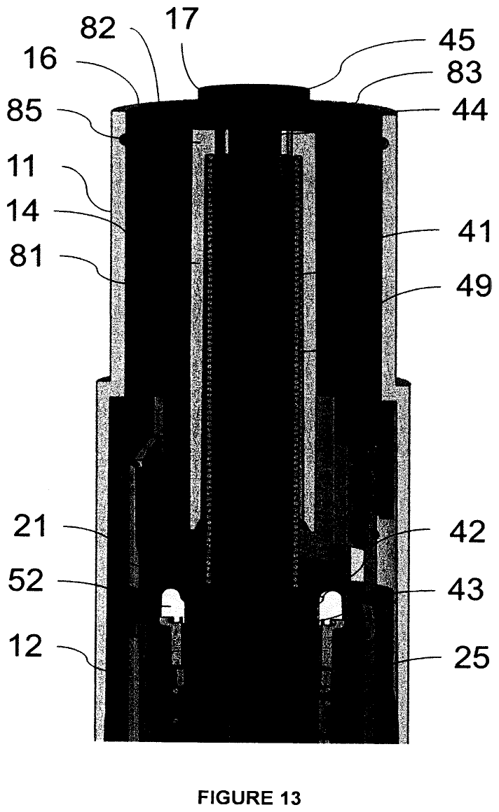

The upper body has a generally cylindrical shape and is open at both ends. The distal end is connected to the lower body. The upper body further comprises an inner cylinder 14 (see FIG. 13) comprising an axial wall parallel to the outer wall of the upper body and connected to said outer wall by a radial wall. The inner cylinder 14 has an open distal end and a partially closed proximal end comprising a radial flange 16 and a central opening 17.

A plunger rod 40 for causing the stopper to move with respect to the medical container 20, as will be explained later, is received within the upper body 11, more precisely within the inner cylinder 14.

The plunger rod 40 comprises a shaft 41 provided at its distal end with a flange 42 and a tip 43 adapted to engage the stopper. The proximal end of the shaft comprises an anti-rotation portion 44 and a toothed disk 45 extending proximally relative to the anti-rotation portion. The shaft 41 may comprise one or more axial rib(s) designed for increasing its stiffness.

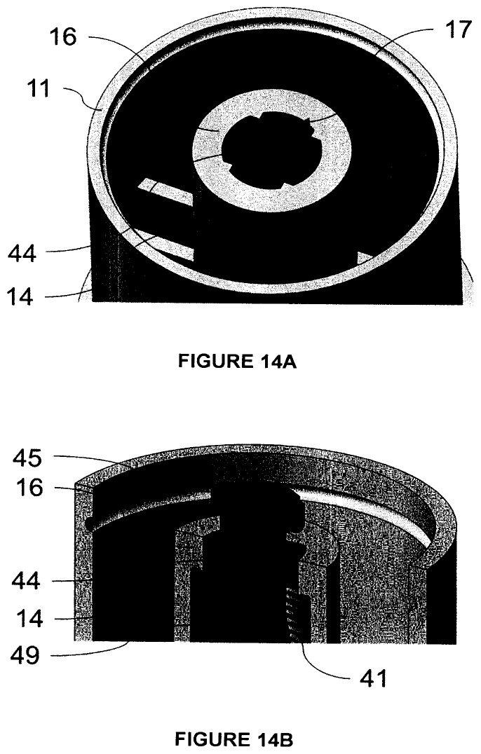

As shown in FIGS. 14A and 14B, the anti-rotation portion 44 has a non-circular shape extending radially outwardly from the shaft. The anti-rotation portion is received in the central opening 17 of the upper body 11 having a substantially complementary shape. As a result, the plunger rod is prevented from rotating relative to the upper body thanks to the engagement of the anti-rotation portion 44 in the opening 17. For example, the anti-rotation portion can comprise at least one flat surface bearing onto a corresponding flat surface of the opening 17.

The toothed disk 45 comprises a plurality of teeth 450 extending radially outwardly and distributed in a regular manner about the axis of the plunger rod.

A first spring 49 is arranged around the shaft 41 of the plunger rod, the distal end of the spring 49 bearing onto the flange 42 and the proximal end of the spring 49 bearing onto an inner surface of the proximal end 16 of the inner cylinder 14 of the upper body 11. In the initial position, the spring is in a compressed state. As a result, the spring 49 urges the plunger rod 40 in the distal direction. However, in the initial position, the plunger rod is locked by a plunger retainer 80 and thus is prevented from translating in the distal direction.

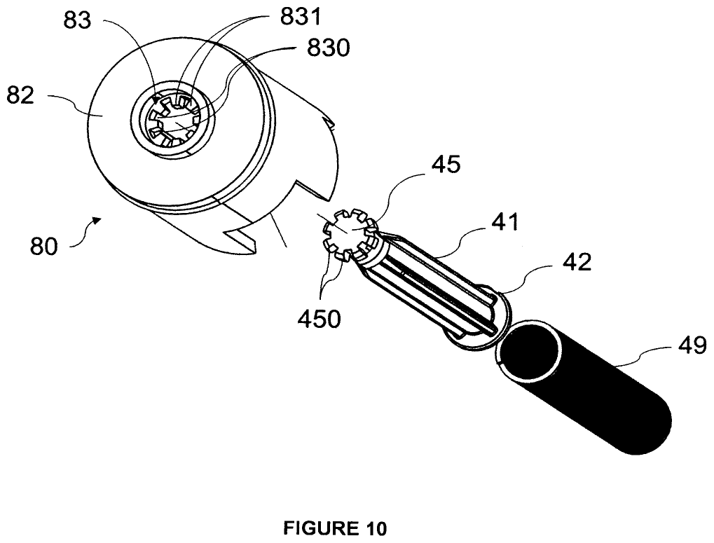

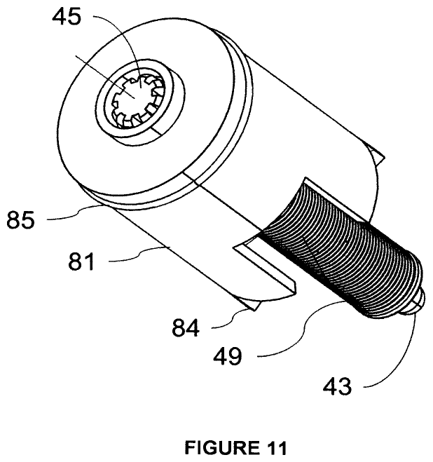

The plunger retainer 80 is pivotally mounted in the upper body 11, in a proximal portion of said upper body. As shown in FIGS. 10 and 11, the plunger retainer comprises an axial skirt 81 and a proximal end 82 comprising a central opening 83. The plunger retainer 80 is blocked in the axial direction relative to the upper body. For example, the plunger retainer 80 comprises a circumferential rim 85 extending radially outwardly from the skirt 81, inserted into a corresponding annular groove in the inner wall of the upper body. The dimensions of the circumferential rim and the annular groove are chosen so as allow rotation of the plunger retainer relative to the upper body.

The central opening 83 of the plunger retainer comprises a plurality of teeth 831 extending radially inwardly.

The central opening 83 extends proximally from the opening 17 of the inner cylinder of the upper body.

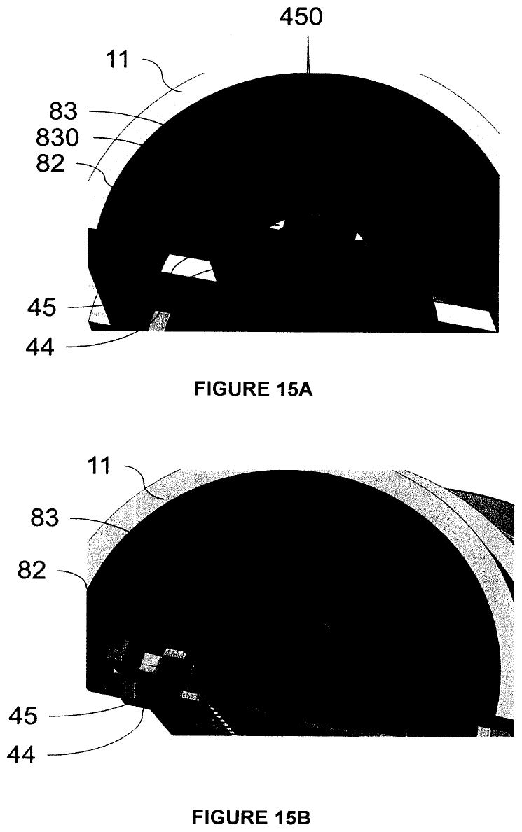

In the initial position (see FIG. 15A), the plunger rod 40 is received in the upper body/plunger retainer assembly such that the anti-rotation portion is received in the opening 17 and the toothed disk 45 of the plunger retainer is located proximally relative to the opening 83 of the plunger retainer. The distal surface of the teeth of the plunger retainer 450 may bear onto the proximal surface of the teeth 831 of the opening 83 of the plunger retainer, which thus prevent the plunger rod from moving axially.

However, the teeth 831 of the plunger retainer and the teeth 450 of the plunger rod as designed to be substantially complementary in shape, i.e. the recesses 830 between the teeth 831 of the plunger retainer 80 substantially fit the teeth 450 of the plunger rod 40, or are greater than said teeth 450 so that the toothed disk 45 of the plunger rod is able to pass through the opening 83 when the teeth 450 of the plunger rod are axially aligned with a corresponding recess 830 between teeth 831 of the central opening 83.

Otherwise said, in some angular positions of the plunger rod relative to the plunger retainer, the plunger rod is locked in distal translation by the plunger retainer (as shown in FIG. 15A), and in other angular positions, the plunger retainer unlocks the plunger rod (as shown in FIG. 15B) and allows it to move distally under the force of the spring 49.

Of course, any other non-circular shapes of the proximal end of the plunger rod and of the plunger retainer could be used, provided that they are complementary.

The skirt 81 of the plunger retainer is provided with at least one distal leg 84, preferably two legs diametrically opposite to each other. The function of said legs will be explained in more detail below.

As shown in FIG. 6, the medical container 20 is mounted in a supporting ring 50. The ring may comprise a distal rigid portion 51 and a proximal elastomeric portion 52. The barrel of the medical container is inserted into the supporting ring 50 so that the proximal flange bears onto the proximal surface of the proximal elastomeric portion 52. The medical container may be maintained in a fixed position relative to the ring by frictional engagement between the elastomeric portion 52 and the barrel.

The ring 50 comprises at least one finger 53 protruding radially from the rigid portion 51. Preferably, the ring comprises two fingers 53 diametrically opposite to each other.

The lower body forms a housing which receives at least partially the medical container 20 and the ring 50. As will appear later, the medical container 20 is movable relative to the lower body between an initial position, in which the tip of the needle does not extend beyond the distal end of the lower body, and an injection position, distally spaced relative to said initial position and in which the tip of the needle extends beyond the distal end of the lower body and is exposed over a predetermined length.

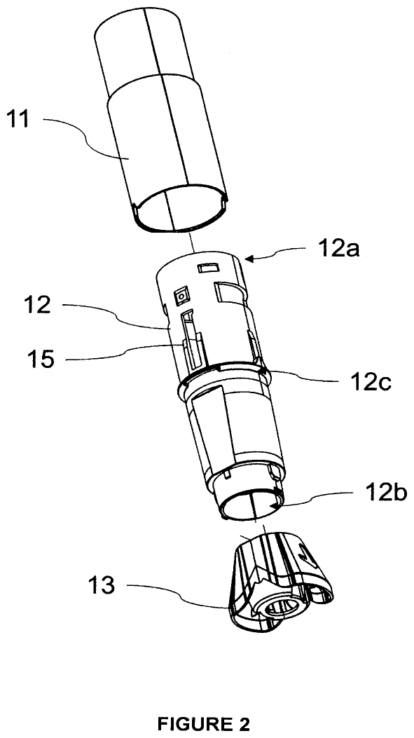

The lower body has a general cylindrical shape and is open at both ends. As shown in FIG. 2, the lower body 12 has a distal part 12b and a proximal part 12a, the diameter of the proximal part 12a being greater than the diameter of the distal part 12b. The proximal part 12a and the distal part 12b are joined by a radial wall 12c. The proximal surface of the radial wall 12c forms an abutment surface for the ring 50.

The lower body comprises at least one axial groove 15 receiving a respective finger 53 of the ring 50. Preferably, the lower body has two axial grooves 15 diametrically opposite to each other, and the ring has two fingers, each received in a respective axial groove. As will be explained in more detail below, such axial groove serves as a guide for axial movement of the ring (and the container) relative to the lower body. Advantageously, said groove is located in a proximal portion of the lower body 12, which is covered by the upper body 11; thus, the groove 15 and the finger 53 are not accessible by a user from the outside of the injection device.

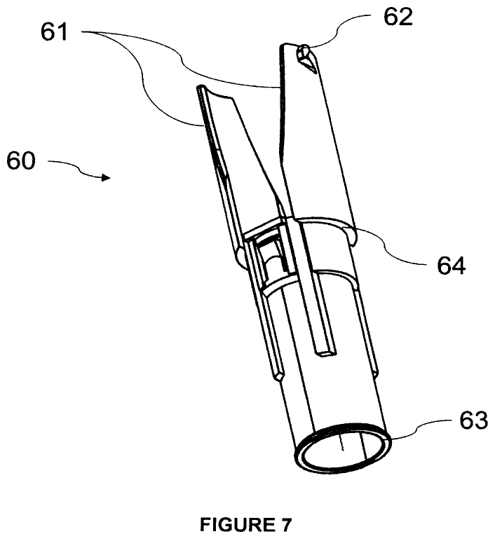

The injection device also includes a safety shield 60 that is at least partially received within a distal portion of the lower body. As shown in FIG. 7, the safety shield 60 comprises two opposite tongues 61 extending proximally. Each tongue 61 comprises a lug 62 extending radially outwardly.

The distal end of the safety shield 60 is advantageously provided with a radial flange 63 forming a bearing surface for applying the injection device onto the patient's skin, the width of the flange being chosen so as to distribute the pushing force on a sufficiently large surface for not injuring the patient.

The safety shield is coupled to a cam 70 pivotably mounted in the lower body 12.

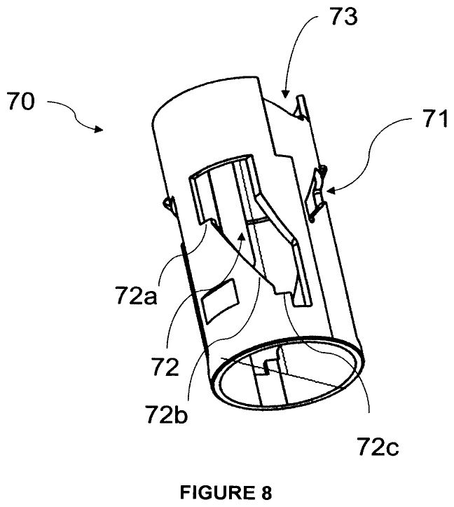

As shown in FIG. 8, the cam 70 has a generally cylindrical shape and is open at both ends.

The cam 70 presents at least one first groove 71, preferably two first grooves diametrically opposite to each other.

Each first groove 71 comprises an inclined (i.e. non-axial) first branch 71a and an axial second branch 71b connected to the first branch at a proximal end of both branches 71a, 71b. Otherwise said, each first groove has the shape of number “1”.

Each lug 62 of the safety shield slidingly engages a respective first groove 71 in the cam 70.

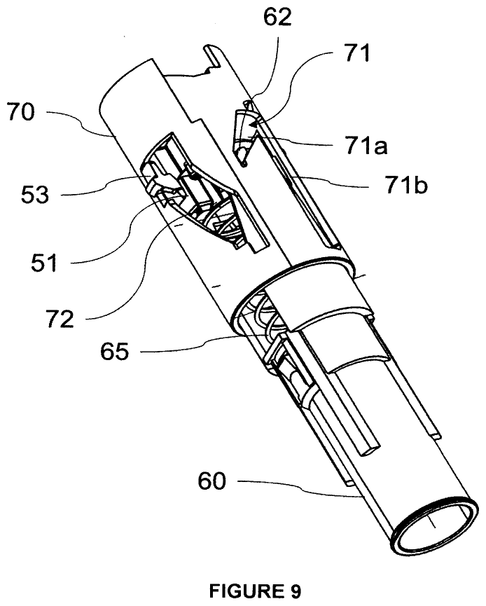

In the initial position, the lug 62 is located at the distal end of the first branch 71a (see FIG. 9) and moves towards the proximal end of the first branch 71a when the safety shield 60 moves from the initial position to the injection position. As a result, such a movement of the safety shield 60 in the proximal direction causes the cam to rotate by an angle depending on the slope of the first branch 71a and the stroke of the lug 62.

When the lug 62 reaches the proximal end of the first branch 71a and engages the second branch 71b, the plunger retainer unlocks the plunger rod, as will be explained below.

A second spring 65 is arranged between the proximal end of the rigid portion of the ring 50 and the flange 64 of the safety shield. In the initial position, the second spring 65 may be in a relaxed state, but a movement of the safety shield 60 in the proximal direction compresses the second spring 65. As a result, if the user releases the pressure applied onto the injection device, the second spring 65 urges the safety shield 60 in the distal direction.

The cam 70 further comprises at least one second groove 72 (preferably two second grooves 72 diametrically opposite to each other). Each second groove 72 comprises a proximal portion 72a extending perpendicular to the direction of injection and a distal portion 72c parallel to the proximal portion, and an inclined (i.e. non-axial) portion 72b connecting the proximal portion 72a to the distal portion 72c. Otherwise said, the proximal and distal portions 72a, 72c of each second groove are spaced both in the axially and angularly. The axial distance between the proximal portion 72a and the distal portion 72c of the second groove 72 is equal to the penetration depth of the needle within the patient's body.

The ring 50 is at least partially received within the cam with each finger 53 of the ring being in sliding engagement within a respective second groove 72.

In the initial position, each finger 53 of the ring is in the proximal portion 72a of the respective second groove 72.

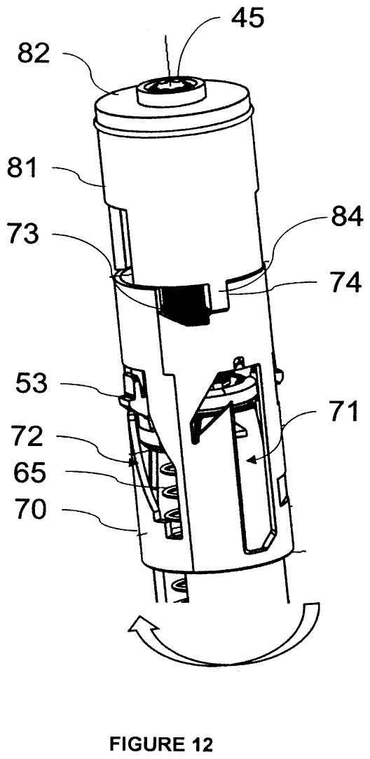

As shown in FIG. 12, the proximal end of the cam 70 comprises at least one recess 73 adapted to receive a respective leg 84 of the plunger retainer. In the initial position, said leg 84 of is in contact with an axial edge 74 of the recess so that, when the cam rotates in the direction indicated by the arrow by the movement of the safety shield 60 in the proximal direction along the first branch 71a of the first groove of the cam (as explained above), the plunger retainer is also caused to rotate in the same direction. However, said rotation of the cam 70 does not cause any movement of the ring 50.

The functioning of the injection device 1 will now be explained.

The injection device is provided to a user ready-to-use, with the cap closing the distal end of the body.

The medical container is filled with a predetermined dose of an injectable product-preferably a single dose thus providing a one-time use or disposable injection device.

Prior to use, the user removes the cap and the needle shield, without rotation of said needle shield. The injection device is thus in its initial position.

The user then places the safety shield against the patient's skin at an injection site. The patient may be the user or another person.

As the device is pressed against the patient's skin, the safety shield is caused to move in the proximal direction and into the lower body.

Due to the above-described safety features, the user cannot activate the device (i.e., cause the container to move from its initial position to its injection position and cause the plunger to push the stopper within the barrel) until the safety shield is caused to move a predetermined distance in the proximal direction so as to allow the plunger retainer driven by the cam to unlock the plunger rod

When the device is pressed against the patient's skin and the safety shield is moved out of its initial position in the proximal direction until the plunger rod is unlocked, the device is automatically activated to begin an injection.

This unlocking of the plunger rod causes the plunger rod to push the stopper in the distal direction. Said movement of the stopper thus causes a movement of the whole medical container in the distal direction from its initial position to its injection position, which also causes the needle to pierce the patient's skin.

Then, the plunger rod is still pushing the stopper in the distal direction in the barrel, which causes the injectable product to automatically be expelled from the container and into the patient's skin.

It is to be noted that, once the plunger retainer is in the suitable position for unlocking the plunger rod, the unlocking of the plunger rod, the movement of the stopper and of the medical container happen during a continuous injection phase, without any further action from the user. In addition, the second position of the safety shield and the unlocking position of the cam are not stable positions but merely intermediate (temporary) positions reached during the triggering of the injection device.

Once the injection is complete, the user removes the device from the injection site and the safety shield is caused to automatically extend from the lower body to cover the now-contaminated tip of the needle. Advantageously, even if the user removes the device from the injection site before the injection is complete, the safety shield will automatically extend over the tip of the needle. Once the injection device is removed from the injection site and the safety shield is extended over the tip of the needle, the shield is locked in place and cannot thereafter be moved from its locked position in the proximal direction to expose the tip of the needle. The used injection device is thus rendered safe for handling and disposal.

The triggering of the injection device thus requires only one action from the user after the cap has been removed.

Referring to the figures, when the user applies the injection device on the injection site by means of the bearing surface 63 of the safety shield 60, a distal force is exerted on the lower body 12 thereby causing the safety shield 60 to move relative to said lower body from the initial position, to a second position in which the plunger retainer driven by the cam unlocks the plunger rod, the second position of the safety shield being proximally spaced relative to said initial position.

During this proximal translation of the safety shield 60 to its second position, each lug 62 translates along the inclined branch 71a of the cam 70, thereby causing the cam 70 to rotate until the connection with the axial branch 71b, which corresponds to the unlocking position of the cam.

If the user releases the pushing force before reaching the second position, the compressed second spring 65 causes the safety shield to move back to the distal position in order to protect the needle.

The rotation of the cam 70 is transmitted to the plunger retainer via the legs 84. The angular displacement of the plunger retainer is chosen so as to align the teeth of the plunger rod with the recesses between the teeth of the opening 83 of the plunger retainer, thereby allowing the toothed disk 45 to pass through the opening 83.

As a result, the plunger rod 40 is caused to move in the distal direction under the distal force of the spring 49.

The distal end 43 of the plunger rod 40 engages the stopper and pushes it in the distal direction. The stopper which is in frictional engagement with the barrel thus causes the whole medical container to move in the distal direction. Since the medical container is supported by the ring 50, the ring 50 is caused to translate in the distal direction with the medical container. During said translation, the fingers 53 of the ring 50 move within the second groove 72 of the cam. More precisely, each finger 53 engages the inclined portion 72b of the respective second groove 72, until the finger 53 reaches the distal portion 72c of the second groove, which prevents any further movement of the ring 50 and the medical container in the distal direction. Said translation of the ring 50 causes the cam 70 to further rotate, which allows the lug 62 to engage the second branch 71b of the first groove 71.

The medical container has thus reached its injection position. In this position, the needle protrudes from the distal end of the lower body 12 and pierces the patient's skin. As noted above, the axial stroke of the finger 53 between the proximal and distal portions 72a, 72c of the second groove 72 of the cam 70 defines the penetration depth of the needle. This ensures that the product be injected at the right depth between the patient's body.

The plunger rod continues pushing the stopper to expel the product from the barrel through the needle until the stopper reaches the distal end of the barrel.

Once the injection is completed, the user withdraws the injection device from the patient's skin.

This withdrawal causes the second spring 65 to push the safety shield 60 in the distal direction, the lugs 62 sliding within the second branch 71b of the first groove 71 of the cam 70, until a final position in which the safety shield covers and protects the needle. When in said final position, the safety shield is locked against proximal movement thereby preventing unintended access to the contaminated needle.

The injection device is thus very easy to use, even in emergency situations, and very safe since it prevents accidental needlestick injuries even in case said device is removed from the injection site before the injection of the product is actually completed.

1. An automatic injection device comprising:
a body;
a medical container comprising a barrel containing a product for injection, a distal tip provided with an injection needle and a stopper in sliding engagement within the barrel, the medical container being slidably arranged in the body between a proximal initial position and a distal position;
a safety shield slidably mounted within the body between a distal initial position and a proximal second position; and
a plunger rod slidably arranged in the body, comprising a non-circular proximal end releasably locked in a proximal initial position and a distal tip adapted to push the stopper in the distal direction when the proximal end is unlocked;
a cam cooperating with the safety shield so that a translation of the safety shield from the distal initial position to the proximal second position causes the cam to rotate within the body to an unlocking position; and
a plunger retainer pivotally mounted in the body, comprising a non-circular proximal opening arranged distally from the proximal end of the plunger rod in the initial position, said opening and the proximal end of the plunger rod having substantially complementary shapes, the plunger retainer being configured to cooperate with the cam so that, in the initial position, the shapes of the proximal end of the plunger rod and of the proximal opening are angularly offset from each other and, in the unlocking position, the shapes of the proximal end of the plunger rod and of the proximal opening are axially aligned, thereby allowing the plunger rod to move in the distal direction through the proximal opening of the plunger retainer.

2. The automatic injection device of claim 1, wherein the proximal end of the plunger rod comprises a plurality of teeth extending radially outwardly and the opening of the plunger retainer comprises a plurality of teeth extending radially inwardly such that the teeth of the plunger rod substantially fit respective recesses between the teeth of the opening of the plunger retainer such that, in the unlocking position, the proximal end of the plunger rod is allowed to pass through the opening of the plunger retainer.

3. The automatic injection device of claim 1, wherein the plunger rod comprises a non-circular anti-rotation portion arranged distally from the proximal end and the body comprises a non-circular central opening, said opening and the anti-rotation portion having substantially complementary shapes so that, in the initial position, the plunger rod is locked in rotation relative to the body by engagement of the anti-rotation portion in the central opening.

4. The automatic injection device of claim 1, wherein the cam comprises at least one recess adapted to receive a respective leg of the plunger retainer against an axial edge of the recess such that a rotation of the cam causes the plunger retainer to rotate.

5. The automatic injection device of claim 1, wherein the safety shield comprises at least one lug received in a respective first groove of the cam so that a movement of the safety shield in the proximal direction causes the cam to rotate.

6. The automatic injection device of claim 5, wherein the first groove presents an inclined first branch and an axial second branch connected to the first branch at a proximal end, such that, in the initial position, the lug is located at the distal end of the first branch and moves towards the proximal end of the first branch when the safety shield moves from the initial position to the second position.

7. The automatic injection device of claim 1, further comprising a first spring configured to urge the plunger rod in the distal direction.

8. The automatic injection device of claim 1, further comprising a second spring configured to urge the safety shield in the distal direction.

9. The automatic injection device of claim 1, further comprising a ring supporting the medical container, the ring comprising at least one finger projecting radially outwardly, the cam further comprising at least one second groove receiving the respective at least one finger, the at least one second groove comprising a proximal portion, a distal portion and an inclined portion connecting the proximal portion to the distal portion,
wherein the ring is locked at the proximal portion of the second groove and moves along the inclined portion to the distal portion when the plunger rod pushes the medical container from the initial position to the injection position.

10. The automatic injection device of claim 9, wherein an axial distance between the proximal portion and the distal portion of the second groove is equal to a penetration depth of the needle within the patient's body.

11. The automatic injection device of claim 9, wherein the at least one finger engages an axial groove of the body.

12. The automatic injection device of claim 9, wherein the first groove presents an inclined first branch and an axial second branch connected to the first branch at a proximal end, such that, in the initial position, the lug is located at the distal end of the first branch and moves towards the proximal end of the first branch when the safety shield moves from the initial position to the second position, and a translation of the ring in the distal direction causes the cam to rotate so that the lug of the safety shield engages the second branch of the first groove of the cam.
